(19) United States
(12) Patent Application Publication
(10) Pub. No.: US 2026/0103695 A1
(43) Pub. Date: Apr. 16, 2026

(54) Optimized SPCAS9 Proteins for Efficient Genome Editing in Eukaryotic Cells

(71) Applicant: Integrated DNA Technologies, Inc., Coralville, IA (US)

(72) Inventors: Christopher Anthony VAKULSKAS, North Liberty, IA (US); Michael Allen COLLINGWOOD, Coralville, IA (US)

(73) Assignee: Integrated DNA Technologies, Inc., Coralville, IA (US)

(21) Appl. No.: 19/046,594

(22) Filed: Feb. 6, 2025

Related U.S. Application Data
(60) Provisional application No. 63/550,226, filed on Feb. 6, 2024.

(51) Int. Cl. C12N 9/22; C12N 15/11

(52) U.S. Cl. CPC C12N 9/226; C12N 15/111; C07K 2319/09; C12N 2310/20

(57) ABSTRACT

This invention pertains to Cas9-NLS fusion proteins and nucleic acids encoding Cas9-NLS fusion proteins for use in CRISPR/Cas endonuclease systems, and their methods of use.

This application claims benefit of U.S. Ser. No. 63/550,226 filed Feb. 6, 2024, the entirety of which is incorporated herein by reference.

### SEQUENCE LISTING

The instant application contains a Sequence Listing that has been submitted in XML format via Patent Center and is hereby incorporated by reference in its entirety. The XML copy, created on Feb. 5, 2025, is named 6391-0021WO01, and is 722,628 bytes in size.

### FIELD OF THE INVENTION

This invention pertains to Cas9 mutant genes, polypeptides encoded by the same and their use in compositions of CRISPR-Cas systems.

### BACKGROUND OF THE INVENTION

The use of clustered regularly interspaced short palindromic repeats (CRISPR) and associated Cas proteins (CRISPR-Cas system) for site-specific DNA cleavage has shown great potential for a number of biological applications. CRISPR is used for genome editing; the genome-scale-specific targeting of transcriptional repressors (CRISPRi) and activators (CRISPRa) to endogenous genes; and other applications of RNA-directed DNA targeting with Cas enzymes.

CRISPR-Cas systems are native to bacteria and Archaea and provide adaptive immunity against viruses and plasmids. Three classes of CRISPR-Cas systems could potentially be adapted for research and therapeutic reagents. Type-II CRISPR systems have a desirable characteristic in utilizing a single CRISPR associated (Cas) nuclease (specifically Cas9) in a complex with the appropriate guide RNAs (gRNAs). In bacteria or Archaea, Cas9 guide RNAs comprise 2 separate RNA species. A target-specific CRISPR-activating RNA (crRNA) directs the Cas9/gRNA complex to bind and target a specific DNA sequence. The crRNA has 2 functional domains, a 5′-domain that is target specific and a 3′-domain that directs binding of the crRNA to the transactivating crRNA (tracrRNA). The tracrRNA is a longer, universal RNA that binds the crRNA and mediates binding of the gRNA complex to Cas9. Binding of the tracrRNA induces an alteration of Cas9 structure, shifting from an inactive to an active conformation. The gRNA function can also be provided as an artificial single guide RNA (sgRNA), where the crRNA and tracrRNA are fused into a single species (see Jinek, M., et al., Science 337 p816-21, 2012). The sgRNA format permits transcription of a functional gRNA from a single transcription unit that can be provided by a double-stranded DNA (dsDNA) cassette containing a transcription promoter and the sgRNA sequence. In mammalian systems, these RNAs have been introduced by transfection of DNA cassettes containing RNA Pol III promoters (such as U6 or H1) driving RNA transcription, viral vectors, and single-stranded RNA following in vitro transcription (see Xu, T., et al., Appl Environ Microbiol, 2014. 80 (5): p. 1544-52).

In the CRISPR-Cas system, using the system present in Streptococcus pyogenes as an example (S.py. or Sp), native crRNAs are about 42 bases long and contain a 5′-region of about 20 bases in length that is complementary to a target sequence (also referred to as a protospacer sequence or protospacer domain of the crRNA) and a 3′ region typically of about 22 bases in length that is complementary to a region of the tracrRNA sequence and mediates binding of the crRNA to the tracrRNA. A crRNA:tracrRNA complex comprises a functional gRNA capable of directing Cas9 cleavage of a complementary target DNA. The native tracrRNAs are about 85-90 bases long and have a 5′-region containing the region complementary to the crRNA. The remaining 3′ region of the tracrRNA includes secondary structure motifs (herein referred to as the “tracrRNA 3′-tail”) that mediate binding of the crRNA:tracrRNA complex to Cas9.

Jinek et al. extensively investigated the physical domains of the crRNA and tracrRNA that are required for proper functioning of the CRISPR-Cas system (Science, 2012. 337 (6096): p. 816-21). They devised a truncated crRNA:tracrRNA fragment that could still function in CRISPR-Cas wherein the crRNA was the wild type 42 nucleotides and the tracrRNA was truncated to 75 nucleotides. They also developed an embodiment wherein the crRNA and tracrRNA are attached with a linker loop, forming a single guide RNA (sgRNA), which varies between 99-123 nucleotides in different embodiments.

At least three groups have elucidated the crystal structure of Streptococcus pyogenes Cas9 (SpCas9). In Jinek, M., et al., the structure did not show the nuclease in complex with either a guide RNA or target DNA. They carried out molecular modeling experiments to reveal predictive interactions between the protein in complex with RNA and DNA (Science, 2014. 343, p. 1215, DOI: 10.1126/science/1247997).

In Nishimasu, H., et al., the crystal structure of Sp Cas9 is shown in complex with sgRNA and its target DNA at 2.5 angstrom resolution (Cell, 2014. 156 (5): p. 935-49, incorporated herein in its entirety). The crystal structure identified two lobes to the Cas9 enzyme: a recognition lobe (REC) and a nuclease lobe (NUC). The sgRNA:target DNA heteroduplex (negatively charged) sits in the positively charged groove between the two lobes. The REC lobe, which shows no structural similarity with known proteins and therefore likely a Cas9-specific functional domain, interacts with the portions of the crRNA and tracrRNA that are complementary to each other.

Another group, Briner et al. (Mol Cell, 2014. 56 (2): p. 333-9, incorporated herein in its entirety), identified and characterized the six conserved modules within native crRNA:tracrRNA duplexes and sgRNA. Anders et al. (Nature, 2014, 513 (7519) p. 569-73) elucidated the structural basis for DNA sequence recognition of protospacer associate motif (PAM) sequences by Cas9 in association with an sgRNA guide.

The CRISPR-Cas endonuclease system is utilized in genomic engineering as follows: the gRNA complex (either a crRNA:tracrRNA complex or an sgRNA) binds to Cas9, inducing a conformational change that activates Cas9 and opens the DNA binding cleft, the protospacer domain of the crRNA (or sgRNA) aligns with the complementary target DNA and Cas9 binds the PAM sequence, initiating unwinding of the target DNA followed by annealing of the protospacer domain to the target, after which cleavage of the target DNA occurs. The Cas9 contains two domains, homologous to endonucleases HNH and RuvC respectively, wherein the HNH domain cleaves the DNA strand complementary to the crRNA and the RuvC-like domain cleaves the non-complementary strand. This results in a double-stranded break in the genomic DNA. When repaired by non-homologous end joining (NHEJ) the break is typically repaired in an imprecise fashion, resulting in the DNA sequence being shifted by 1 or more bases, leading to disruption of the natural DNA sequence and, in many cases, leading to a frameshift mutation if the event occurs in a coding exon of a protein-encoding gene. The break may also be repaired by homology directed recombination (HDR), which permits insertion of new genetic material based upon exogenous DNA introduced into the cell with the Cas9/gRNA complex, which is introduced into the cut site created by Cas9 cleavage.

Certain improvements in the utility of Cas9 has been achieved by enhancing its delivery into the eukaryotic nucleus through the use of improved nuclear localization signals (NLS; Kalderon, D., Roberts, B. L., Richardson, W. D. & Smith, A. E. A short amino acid sequence able to specify nuclear location. Cell 39, 499-509 (1984).) Currently, the canonical SV40 NLS is commonly added to the amino- or carboxy-terminus to facilitate active transport of proteins into the eukaryotic nucleus. The SV40 NLS is viral in origin and demonstrates strong activity in a wide variety of different eukaryotic species including both plants and animals. Though the SV40 NLS has been used with wide success, other variants or mutants with higher intrinsic localization activity have been described. Typically, the context and linkage of an NLS with respect to the target fusion protein is also important and can determine the overall improvement in nuclear localization: there appears to be no “one size fits all” NLS solution for all proteins.

The prior art consists of using the native Cas9 enzyme absent optimized nuclear localization tags, of which only a small fraction of material can spontaneously enter the nuclear envelope resulting in poor editing efficiency. Thus, there is a long-felt need to provide Cas9 fusion proteins linked to improved NLS motifs that further enhance the nuclear localization of the CRISPR/Cas9 ribonucleoprotein complex, thereby improving CRISPR/Cas9 editing activity.

### BRIEF SUMMARY OF THE INVENTION

This invention pertains to Cas9-NLS fusion proteins and nucleic acids that encode the same for use in CRISPR systems, and their methods of use.

In a first aspect, an amino acid sequence including a nuclear localization signal (NLS) is provided. The amino acid sequence of the NLS includes a member selected from the group consisting of SEQ ID NO: 4, 11, 12, 13, 14, 15, 16, 17, 18, 19, 23, 26, 31, 33, 34, 38, 39, 40, 41, 42, 43, 44, 46, 47, 48, 49, 50, 51, 52, 55, 57, 58, 59, 60, 62, 63, 65, 66, 67, 69, 70, 71, 72, 74, 75, 77, 78, and 79.

In a second aspect, a fusion protein including a first amino acid sequence and a second amino acid sequence is provided. The second amino acid sequence includes a nuclear localization signal (NLS), wherein the NLS includes a member selected from the group consisting of SEQ ID NO: 4, 11, 12, 13, 14, 15, 16, 17, 18, 19, 23, 26, 31, 33, 34, 38, 39, 40, 41, 42, 43, 44, 46, 47, 48, 49, 50, 51, 52, 55, 57, 58, 59, 60, 62, 63, 65, 66, 67, 69, 70, 71, 72, 74, 75, 77, 78, and 79.

In a third aspect, an isolated Cas9-NLS fusion protein is provided. The Cas9-NLS fusion protein includes a Cas9 amino acid sequence and a nuclear localization signal sequence (NLS). The isolated Cas9-NLS fusion protein is active in a CRISPR/Cas endonuclease system, wherein the isolated Cas9-NLS fusion protein comprises a member selected from the group consisting of SEQ ID NO:162-239.

In a fourth aspect, a nucleic acid sequence encoding a nuclear localization signal (NLS) is provided. The nucleic acid sequence encoding the NLS includes a member selected from the group consisting of SEQ ID NO: 83, 90, 91, 92, 93, 94, 95, 96, 97, 98, 102, 105, 110, 113, 117, 118, 119, 120, 121, 122, 123, 125, 126, 127, 128, 129, 130, 131, 134, 136, 137, 138, 139, 141, 142, 144, 145, 146, 148, 149, 150, 151, 153, 154, 156, 157, and 158.

In a fifth aspect, an isolated nucleic acid sequence encoding the isolated Cas9-NLS fusion protein is provided.

In a sixth aspect, an isolated ribonucleoprotein complex is provided. The isolated ribonucleoprotein complex includes a Cas9-NLS fusion protein and a gRNA.

In a seventh aspect, a CRISPR/Cas endonuclease system is provided. The CRISPR/Cas endonuclease system includes an isolated Cas9-NLS fusion protein. The isolated Cas9-NLS fusion protein includes a member selected from the group consisting of SEQ ID NO: 162-239.

In an eighth aspect, a method of performing gene editing in a eukaryotic cell is provided. The method includes a step of contacting a candidate editing target site locus with an active CRISPR/Cas endonuclease system having a Cas9-NLS fusion protein. The Cas9-NLS fusion protein includes a member selected from the group consisting of SEQ ID NO: 162-239.

In a ninth aspect, a kit for performing gene editing in a eukaryotic cell is provided. The kit includes a Cas9-NLS fusion protein, optionally, a nucleic acid encoding a Cas9-NLS fusion protein, and, optionally, a gRNA. The Cas9-NLS fusion protein includes a member selected from the group consisting of SEQ ID NO: 162-239. Where included in the kit, the nucleic acid encoding a Cas9-NLS fusion protein includes a member selected from the group consisting of SEQ ID NO: 241-318.

### BRIEF DESCRIPTION OF THE DRAWINGS

FIG. 1A depicts genome editing activity of representative Cas9-NLS fusion proteins of the present invention that were separately delivered by plasmid into HEK293 cells as compared to the genome editing activity of a prior art Cas9-NLS fusion protein (SV40 NLS motif; SEQ ID NO:1) delivered in a similar manner; see Table 2 for a complete description of the SEQ ID NO for the Cas9-NLS fusion protein amino acid sequences and NLS amino acid sequences.

FIG. 1B depicts genome editing activity of representative Cas9-NLS fusion proteins of the present invention that were separately delivered by plasmid into HEK293 cells as compared to the genome editing activity of a prior art Cas9-NLS fusion protein (SV40 NLS motif; SEQ ID NO:1) delivered in a similar manner; see Table 2 for a complete description of the SEQ ID NO for the Cas9-NLS fusion protein amino acid sequences and NLS amino acid sequences.

FIG. 1C depicts genome editing activity of representative Cas9-NLS fusion proteins of the present invention that were separately delivered by plasmid into HEK293 cells as compared to the genome editing activity of a prior art Cas9-NLS fusion protein (SV40 NLS motif; SEQ ID NO:1) delivered in a similar manner; see Table 2 for a complete description of the SEQ ID NO for the Cas9-NLS fusion protein amino acid sequences and NLS amino acid sequences.

FIG. 2A depicts genome editing activity of purified Cas9-NLS fusion proteins of the present invention delivered as RNP complexes into HEK293 cells compared to the genome editing activity of two prior art Cas9-NLS commercial proteins.

FIG. 2B depicts genome editing activity of purified Cas9-NLS fusion proteins of the present invention delivered as RNP complexes into K562 cells compared to the genome editing activity of two prior art Cas9-NLS commercial proteins.

### DETAILED DESCRIPTION OF THE INVENTION

The present invention pertains to a high-throughput plasmid-based screen to identify Cas9-NLS fusion proteins having improvements in nuclear localization in eukaryotic cells, as revealed through increased genome editing. The WT Cas9 sequence was fused to NLS sequences gathered from the published literature, which includes NLS examples from native proteins, entirely synthetic NLS sequences, or NLS sequences that match the known consensus sequences for nuclear transport proteins (i.e., Importin a/B) (Kosugi, S. et al. Six classes of nuclear localization signals specific to different binding grooves of importin alpha. J Biol Chem 284, 478-485 (2009)).

The term “wild-type Cas9 protein” (“WT-Cas9” or “WT-Cas9 protein”) encompasses a protein having the identical amino acid sequence of the naturally-occurring Streptococcus pyogenes Cas9 (e.g., SEQ ID No.: 159) and that has biochemical and biological activity when combined with a suitable guide RNA (for example sgRNA or dual crRNA:tracrRNA compositions) to form an active CRISPR-Cas endonuclease system.

The term “wild-type CRISPR/Cas endonuclease system” refers to a CRISPR/Cas endonuclease system that includes wild-type Cas9 protein and a suitable gRNA.

The phrase “active CRISPR/Cas endonuclease system displays reduced off-target editing activity and maintained on-target editing activity relative to a wild-type CRISPR/Cas endonuclease system” refers to the activity of a CRISPR/Cas endonuclease system that includes a mutant Cas9 protein that displays a reduction in off-targeting editing activity that is typically greater than the reduction in on-target editing activity relative to the corresponding off-target and on-target editing activities of a wild-type CRISPR/Cas endonuclease system that includes wild-type Cas9 protein when both CRISPR/Cas endonuclease systems include the identical gRNA for a given target sequence. Preferred off-target and on-target activities of the CRISPR/Cas endonuclease systems depend upon the gRNA and the target sequence of interest; such preferred off-target and on-target activities of CRISPR/Cas endonuclease systems having mutant Cas9 proteins are illustrated in the Examples.

The term “Cas9-NLS fusion protein” encompasses protein forms having a WT Cas9 amino acid sequence fused to a nuclear localization sequence motif (NLS), wherein the resultant protein displays biochemical and biological activity when combined with a suitable guide RNA (for example sgRNA or dual crRNA:tracrRNA compositions) to form an active CRISPR-Cas endonuclease system.

The term “isolated nucleic acid” include DNA, RNA, cDNA, and vectors encoding the same, where the DNA, RNA, cDNA and vectors are free of other biological materials from which they may be derived or associated, such as cellular components. Typically, an isolated nucleic acid will be purified from other biological materials from which they may be derived or associated, such as cellular components.

The term “isolated wild-type Cas9 nucleic acid” is an isolated nucleic acid that encodes a wild-type Cas9 protein. Examples of an isolated wild-type Cas9 nucleic acid include SEQ ID NO:160.

The term “isolated Cas9-NLS fusion protein nucleic acid” is an isolated nucleic acid that encodes a Cas9-NLS fusion protein.

A competent CRISPR-Cas endonuclease system includes a ribonucleoprotein (RNP) complex formed with isolated Cas9 protein or Cas9-NLS fusion protein and an isolated guide RNA selected from one of a dual crRNA:tracrRNA combination or a chimeric single-molecule sgRNA. In some embodiments, isolated length-modified and/or chemically-modified forms of crRNA and tracrRNA are combined with purified Cas9 protein or Cas9-NLS fusion protein, an isolated mRNA encoding Cas9 protein or Cas9-NLS fusion protein, or a gene encoding Cas9 protein or Cas9-NLS fusion protein in an expression vector. In certain assays, isolated crRNA and tracrRNA can be introduced into cell lines that stably express Cas9 protein or Cas9-NLS fusion protein from an endogenous expression cassette encoding the Cas9 gene or Cas9-NLS fusion protein gene. In other assays, a mixture of crRNA and tracrRNA in combination with either Cas9-NLS fusion protein mRNA or Cas9-NLS fusion protein can be introduced into cells.

### Screening and Identification of Novel Cas9-NLS Fusion Proteins Having Surprisingly Robust Genome Editing Activity

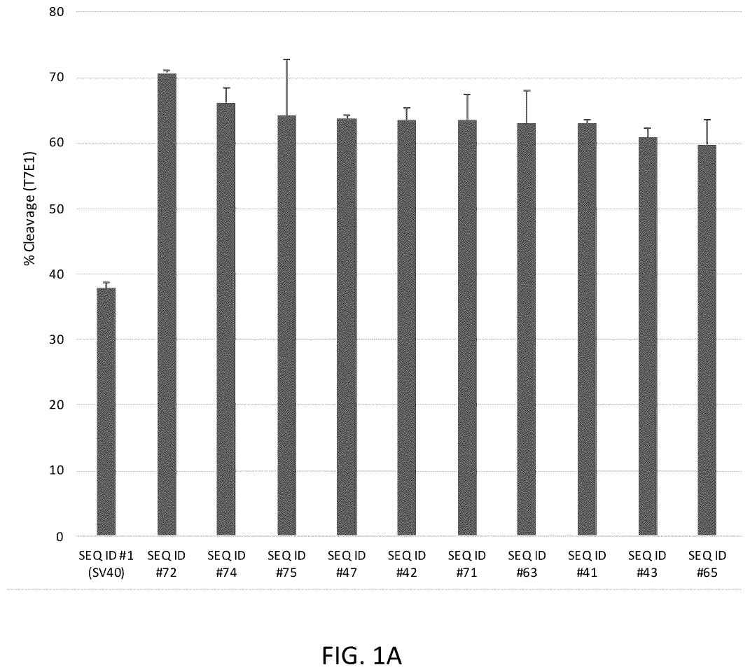

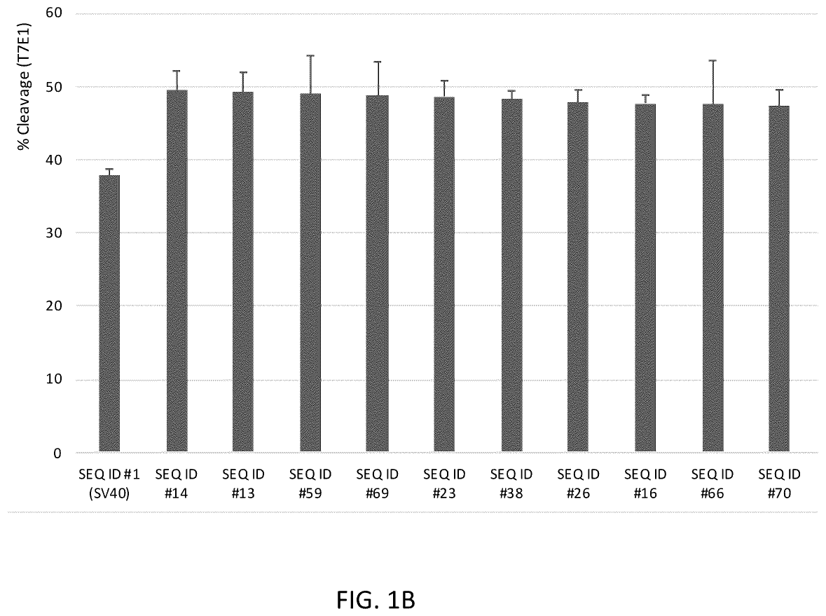

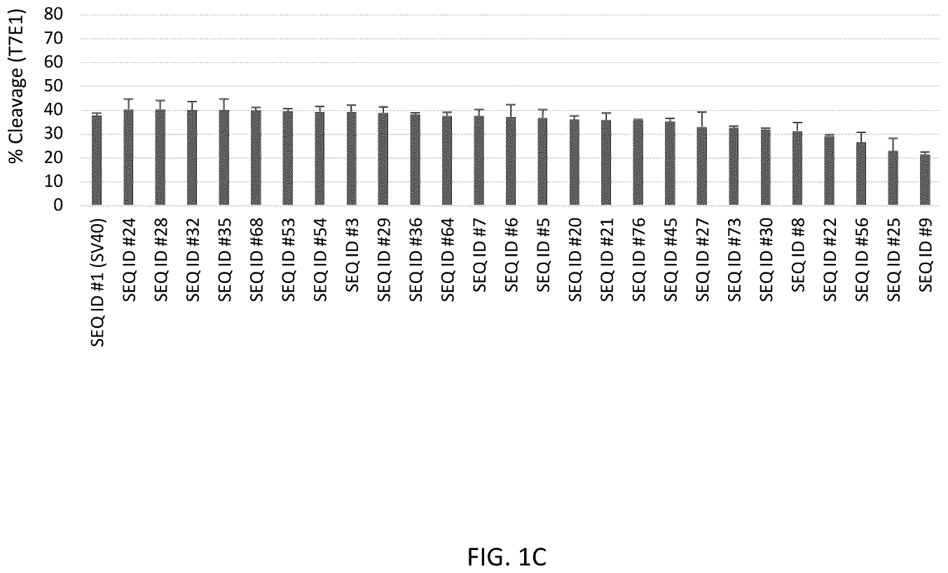

The genome editing phenotype of the SpCas9-NLS fusion proteins described in Table 1 are presented in FIG. 1 and Table 2. Three biological replicates were performed with high consistency, which enabled us to confidently isolate a large collection of novel SpCas9-NLS fusion protein variants with enhanced genome editing activity. We chose the most promising variants from the initial plasmid expression screen and purified these SpCas9-NLS fusion proteins and determined cleavage activity with ribonucleoprotein (RNP) delivery of these variants into human cell lines. The results indicate that our screens have revealed SpCas9-NLS fusions with unexpectedly increased potency over commercially available Cas9-NLS proteins and controls.

TABLE 1
NLS amino acid and nucleotide sequences

| SEQ | | |
|---|---|---|
| ID | | |
| NO.^{a} | AA sequence | DNA sequence |
| 1; 80 | AAPKKKRKV (SV40) | GCGGCCCCCAAGAAAAAGCGGAAGGTGTGA |
| 2; 81 | AAKRKNSATVHLCPVP | GCGGCCAAGAGAAAGAACAGCGCCACCGTGCACCTGTGTCCTGTGCCTAGAA |
| | RKR | AGAGATGA |
| 3; 82 | AAKRPAELDGADNASQ | GCGGCCAAAAGACCCGCCGAACTGGACGGCGCCGATAATGCTTCTCAGGCCG |
| | AAKRR | CCAAGAGAAGATGA |
| 4; 83 | AARKRNSATVHLCPVP | GCGGCCCGGAAGAGAAATAGCGCCACCGTGCACCTGTGTCCTGTGCCTAGAA |
| | RKR | AGAGATGA |
| 5; 84 | AAKRPATLANNSPAAKR | GCGGCCAAGAGGCCTGCCACACTGGCCAACAATTCTCCAGCCGCCAAGCGGA |
| | R | GATGA |
| 6; 85 | AAKRPATLANESPAAKR | GCGGCCAAGAGGCCTGCCACACTGGCCAATGAATCTCCAGCCGCCAAGCGGA |
| | R | GATGA |
| 7; 86 | AARRRNSATVHLCPVP | GCGGCCCGGAGAAGAAATAGCGCCACCGTGCACCTGTGTCCTGTGCCTAGAA |
| | RKR | AGAGATGA |
| 8; 87 | AADTRLGKRKRRPW | GCGGCCGATACCAGACTGGGCAAGAGAAAGAGAAGGCCCTGGTGA |
| 9; 88 | AARRRSVLKRSWSVAF | GCGGCCCGGAGAAGAAGCGTGCTGAAGAGATCTTGGAGCGTGGCCTTCTGA |
| 10; 89 | AAKRPATLANDSPAAKR | GCGGCCAAGAGGCCTGCCACACTGGCCAATGATTCTCCAGCCGCCAAGCGGA |
| | R | GATGA |
| 11; 90 | AAKRPAALAGAGNASQ | GCGGCCAAAAGACCTGCTGCTCTGGCTGGCGCCGGAAATGCTTCTCAGGCCG |
| | AAKRR | CCAAAAGAAGATGA |
| 12; 91 | AAKRPAELDEADNASQ | GCGGCCAAAAGACCCGCCGAGCTGGACGAAGCCGATAATGCTTCTCAGGCCG |
| | AAKRR | CCAAGCGGAGATGA |
| 13; 92 | AARRRKRRREWEDF | GCGGCCCGGAGAAGAAAGCGGCGCAGAGAGTGGGAAGATTTCTGA |
| 14; 93 | AATSPSRKRKWDQV | GCGGCCACAAGCCCTAGCCGGAAGAGAAAGTGGGACCAAGTGTGA |
| 15; 94 | AAAVKRPAATKKAGQA | GCGGCCGCCGTGAAAAGACCTGCCGCCACAAAGAAAGCCGGCCAGGCCAAGA |
| | KKKKLD | AGAAGAAGCTGGACTGA |
| 16; 95 | AAPILGKRKRHLFL | GCGGCCCCTATCCTGGGCAAGAGAAAGCGGCACCTGTTTCTGTGA |
| 17; 96 | AAPAAKRVKLD | GCGGCCCCTGCCGCCAAGAGAGTGAAGCTGGATTGA |
| 18; 97 | AAMSRRRKANPTKLSE | GCGGCCATGAGCAGACGGCGGAAGGCCAATCCTACCAAGCTGAGCGAGAACG |
| | NAKKLAKEVEN | CCAAGAAACTGGCCAAAGAGGTGGAAAACTGA |
| 19; 98 | AATLGKRKRISCVT | GCGGCCACCCTGGGAAAGAGAAAGCGGATCAGCTGCGTGACCTGA |
| 20; 99 | AAKLKIKRPVK | GCGGCCAAGCTGAAGATCAAGCGGCCCGTGAAGTGA |
| 21; 100 | AARVLGKRKREDRP | GCGGCCAGAGTGCTGGGCAAGCGCAAGAGAGAGGACAGACCTTGA |
| 22;101 | AALLGKRKRPSIEH | GCGGCCCTGCTGGGCAAGAGAAAGAGGCCTAGCATCGAGCACTGA |
| 23; 102 | AAPVLGKRKRSLSS | GCGGCCCCTGTGCTGGGCAAGAGAAAGAGAAGCCTGAGCAGCTGA |
| 24; 103 | AAILGKRKRSHHPY | GCGGCCATCCTGGGCAAGCGCAAGAGAAGCCACCATCCTTACTGA |
| 25; 104 | AASMLGKRKRCIIS | GCGGCCAGCATGCTGGGCAAGAGAAAGCGGTGCATCATCAGCTGA |
| 26; 105 | AASVLGKRKRHHLD | GCGGCCTCTGTGCTGGGCAAGAGAAAGAGACACCACCTGGACTGA |
| 27; 106 | AATVHLGKRRLRPW | GCGGCCACAGTGCACCTGGGCAAGAGAAGGCTCAGACCTTGGTGA |
| 28; 107 | AASVLGKRKRHPKV | GCGGCCTCTGTGCTGGGCAAGAGAAAGCGGCACCCTAAGGTTTGA |
| 29; 108 | AASILGKRKNRDPS | GCGGCCAGCATCCTGGGCAAGAGAAAGAACAGGGACCCCAGCTGA |
| 30; 109 | AARVLGKRKTGRSP | GCGGCCAGAGTGCTGGGCAAGAGAAAGACCGGCAGAAGCCCTTGA |
| 31; 110 | AAVLGKRKRDDCW | GCGGCCGTGCTGGGCAAGAGAAAGAGAGATGACTGCTGGTGA |
| 32; 111 | AAKRKCAVFLEGQN | GCGGCCAAGAGAAAGTGCGCCGTGTTCCTGGAAGGCCAGAACTGA |
| 33; 112 | AAHGRQVLGKRKR | GCGGCCCATGGTAGACAGGTGCTGGGCAAGAGAAAGCGGTGA |
| 34; 113 | AAVHKTVLGKRKYW | GCGGCCGTGCACAAGACAGTGCTGGGCAAGAGAAAGTACTGGTGA |
| 35; 114 | AAKRKYAVFLESQN | GCGGCCAAGCGGAAGTACGCCGTGTTCCTGGAAAGCCAGAACTGA |
| 36; 115 | AAKRKWMAFVMGDP | GCGGCCAAGAGAAAGTGGATGGCCTTCGTGATGGGCGACCCTTGA |
| 37; 116 | AAIPRKRSFAELYD | GCGGCCATCCCCAGAAAGAGAAGCTTCGCCGAGCTGTACGACTGA |
| 38; 117 | AAWAGRKRTWRDAF | GCGGCCTGGGCCGGAAGAAAAAGAACATGGCGGGACGCCTTCTGA |
| 39; 118 | AARLTPRKRAFSEV | GCGGCCAGACTGACCCCTAGAAAGCGGGCCTTTAGCGAGGTTTGA |
| 40; 119 | AAQSVLGKRKSRPF | GCGGCCCAGTCTGTGCTGGGCAAGAGAAAGAGCAGACCCTTCTGA |
| 41; 120 | AASSHRKRKFSDAF | GCGGCCAGCAGCCACCGGAAGAGAAAGTTCAGCGACGCCTTCTGA |
| 42; 121 | AAPSHRKRKFSDAF | GCGGCCCCCAGCCACCGGAAGAGAAAGTTCAGCGACGCCTTCTGA |
| 43; 122 | AATAHRKRKFSDAF | GCGGCCACAGCCCACCGGAAGAGAAAGTTCAGCGACGCCTTTTGA |
| 44; 123 | AARVQRKRKWSEAF | GCGGCCCGGGTGCAGCGGAAAAGAAAGTGGAGCGAGGCCTTCTGA |
| 45; 124 | AAHRYCGKRRRRTR | GCGGCCCACAGATACTGCGGCAAGCGGCGGAGAAGAACCAGATGA |
| 46; 125 | AATYSGVKRKRNVV | GCGGCCACATACAGCGGCGTGAAGCGGAAGAGAAACGTGGTGTGA |
| 47; 126 | AARLTRKRKYDCAF | GCGGCCCGGCTGACCAGAAAGCGGAAGTACGATTGCGCCTTCTGA |
| 48; 127 | AAKRKYSIYLGSQS | GCGGCCAAGCGGAAGTACAGCATCTACCTGGGCAGCCAGTCTTGA |
| 49; 128 | AALVNRKRRYWEAF | GCGGCCCTGGTCAACCGGAAGCGGAGATACTGGGAAGCCTTCTGA |
| 50; 129 | AATHIGYKRKRDSV | GCGGCCACCCACATCGGCTACAAGAGAAAGCGGGACAGCGTGTGA |
| 51; 130 | AAKKGKRKRLVRPW | GCGGCCAAGAAAGGCAAGCGGAAGAGACTCGTGCGGCCTTGGTGA |
| 52; 131 | AAYGRVSKRPRYQF | GCGGCCTACGGCAGAGTGTCCAAGAGGCCCAGATACCAGTTCTGA |
| 53; 132 | AASVLGKRSRTWE | GCGGCCTCTGTGCTGGGCAAGAGAAGCAGAACCTGGGAGTGA |
| 54; 133 | AATLERKRKLAVLY | GCGGCCACCCTGGAACGGAAGAGAAAACTGGCCGTGCTGTACTGA |
| 55; 134 | AARKRGRKRFRSV | GCGGCCCGGAAGAGAGGCCGGAAGAGATTCAGAAGCGTGTGA |
| 56; 135 | AAQRRLLKRKRGSL | GCGGCCCAGAGAAGGCTGCTGAAGAGAAAGAGAGGCAGCCTGTGA |
| 57; 136 | AAIGRKRGYSVAFG | GCGGCCATCGGCAGAAAGCGGGGCTACTCTGTGGCCTTTGGATGA |
| 58; 137 | AAKRPYSIAFPLGQ | GCGGCCAAGCGGCCCTACTCTATCGCCTTTCCACTGGGACAATGA |
| 59; 138 | AAITRKRKRDLVFT | GCGGCCATCACCCGGAAGAGAAAGCGCGACCTGGTGTTCACATGA |
| 60; 139 | AAKRTWAQAFTE | GCGGCCAAGAGAACATGGGCCCAAGCCTTCACCGAGTGA |
| 61; 140 | AAKRRYSDAFRLPV | GCGGCCAAGCGGAGATACAGCGACGCCTTCAGACTGCCTGTTTGA |
| 62; 141 | AAKRSWSMAFC | GCGGCCAAGCGGTCTTGGAGCATGGCCTTTTGCTGA |
| 63; 142 | AAISRKRKRDLEFV | GCGGCCATCAGCCGGAAGAGAAAGCGCGACCTGGAATTCGTGTGA |
| 64; 143 | AAIGRKRVWAVAFY | GCGGCCATCGGACGGAAAAGAGTGTGGGCCGTCGCCTTCTATTGA |
| 65; 144 | AAKRKYSDAFGLPV | GCGGCCAAGCGGAAGTACAGCGACGCCTTTGGACTGCCTGTTTGA |
| 66; 145 | AAKRKRWENDIP | GCGGCCAAGCGGAAGAGATGGGAGAACGACATCCCCTGA |
| 67; 146 | AAKRKRWENNIP | GCGGCCAAGCGGAAGAGATGGGAGAACAACATCCCCTGA |
| 68; 147 | AATGGVMKRKRGSV | GCGGCCACAGGCGGCGTGATGAAGAGAAAAAGGGGCAGCGTTTGA |
| 69; 148 | AALPKKRKFSEISS | GCGGCCCTGCCCAAGAAGCGGAAGTTCAGCGAGATCAGCAGCTGA |
| 70; 149 | AALSGTKRKRAYFI | GCGGCCCTGAGCGGCACCAAGAGAAAGCGGGCCTACTTCATCTGA |
| 71; 150 | AAPSRKRKRDHYAV | GCGGCCCCCAGCAGAAAGAGAAAGCGGGATCACTACGCCGTGTGA |
| 72; 151 | AAEPNPRKRKRSEL | GCGGCCGAGCCCAATCCTCGGAAGAGAAAGAGAAGCGAGCTGTGA |
| 73; 152 | AAPILPLKRRRGSP | GCGGCCCCTATCCTGCCTCTGAAGAGAAGAAGAGGCAGCCCCTGA |
| 74; 153 | AAQIGKKRKRDYLD | GCGGCCCAGATCGGCAAGAAGCGGAAGAGAGACTACCTGGACTGA |
| 75; 154 | AAPSRKRKRESDHI | GCGGCCCCCTCCAGAAAGCGGAAGAGAGAGAGCGACCACATCTGA |
| 76; 155 | AAKRGKRKRLVRPW | GCGGCCAAGCGGGGCAAGAGAAAAAGACTCGTGCGGCCTTGGTGA |
| 77; 156 | AARSSGILGKRKFE | GCGGCCAGAAGCTCTGGCATCCTGGGCAAGAGAAAGTTCGAGTGA |
| 78; 157 | AAKRPSATVHLCPVPRK | GCGGCCAAAAGACCTAGCGCCACCGTGCACCTGTGTCCTGTGCCT |
| | R | AGAAAGAGATGA |
| 79; 158 | AALGKRYDRDWDYK | GCGGCCCTGGGCAAGAGATACGACAGAGACTGGGACTACAAGTGA |

ªThe first SEQ ID NO corresponds to the amino acid sequence; the second SEQ ID NO corresponds to the nucleic acid sequence.

TABLE 2
Cleavage activity of SpCas9-NLS fusion proteins in HEK293 cells.

| Cas9-NLS Fusion | NLS AA | % Cleavage | |
|---|---|---|---|
| Protein Sequence | Sequence | (Average)^{a} | Std Dev |
| SEQ ID NO: 161 | SEQ ID NO: 1 (SV40) | 37.8 | 0.9 |
| SEQ ID NO232 | SEQ ID NO: 72 | 70.7 | 0.3 |
| SEQ ID NO: 234 | SEQ ID NO: 74 | 66.1 | 2.3 |
| SEQ ID NO: 235 | SEQ ID NO: 75 | 64.1 | 8.6 |
| SEQ ID NO: 207 | SEQ ID NO: 47 | 63.8 | 0.5 |
| SEQ ID NO: 202 | SEQ ID NO: 42 | 63.5 | 1.8 |
| SEQ ID NO: 231 | SEQ ID NO: 71 | 63.5 | 4.0 |
| SEQ ID NO: 223 | SEQ ID NO: 63 | 62.9 | 5.1 |
| SEQ ID NO: 201 | SEQ ID NO: 41 | 62.9 | 0.6 |
| SEQ ID NO: 243 | SEQ ID NO: 43 | 60.8 | 1.4 |
| SEQ ID NO: 225 | SEQ ID NO: 65 | 59.8 | 3.8 |
| SEQ ID NO: 212 | SEQ ID NO: 52 | 59.5 | 1.0 |
| SEQ ID NO: 209 | SEQ ID NO: 49 | 58.2 | 2.6 |
| SEQ ID NO: 175 | SEQ ID NO: 15 | 58.0 | 1.2 |
| SEQ ID NO: 204 | SEQ ID NO: 44 | 57.3 | 3.6 |
| SEQ ID NO: 210 | SEQ ID NO: 50 | 57.2 | 1.0 |
| SEQ ID NO: 237 | SEQ ID NO: 77 | 54.8 | 1.4 |
| SEQ ID NO: 199 | SEQ ID NO: 39 | 54.6 | 0.3 |
| SEQ ID NO: 215 | SEQ ID NO: 55 | 54.4 | 4.1 |
| SEQ ID NO: 220 | SEQ ID NO: 60 | 54.1 | 2.7 |
| SEQ ID NO: 177 | SEQ ID NO: 17 | 54.0 | 0.5 |
| SEQ ID NO: 238 | SEQ ID NO: 78 | 53.8 | 1.5 |
| SEQ ID NO: 200 | SEQ ID NO: 40 | 52.7 | 1.5 |
| SEQ ID NO: 193 | SEQ ID NO: 33 | 52.3 | 4.5 |
| SEQ ID NO: 239 | SEQ ID NO: 79 | 51.7 | 0.3 |
| SEQ ID NO: 218 | SEQ ID NO: 58 | 51.7 | 2.3 |
| SEQ ID NO: 191 | SEQ ID NO: 31 | 49.6 | 2.6 |
| SEQ ID NO: 174 | SEQ ID NO: 14 | 49.5 | 2.6 |
| SEQ ID NO: 173 | SEQ ID NO: 13 | 49.2 | 2.8 |
| SEQ ID NO: 219 | SEQ ID NO: 59 | 49.0 | 5.2 |
| SEQ ID NO: 229 | SEQ ID NO: 69 | 48.7 | 4.6 |
| SEQ ID NO: 183 | SEQ ID NO: 23 | 48.7 | 2.1 |
| SEQ ID NO: 198 | SEQ ID NO: 38 | 48.3 | 1.1 |
| SEQ ID NO: 186 | SEQ ID NO: 26 | 47.7 | 1.8 |
| SEQ ID NO: 176 | SEQ ID NO: 16 | 47.7 | 1.1 |
| SEQ ID NO: 226 | SEQ ID NO: 66 | 47.5 | 6.0 |
| SEQ ID NO: 230 | SEQ ID NO: 70 | 47.3 | 2.2 |
| SEQ ID NO: 206 | SEQ ID NO: 46 | 47.3 | 2.9 |
| SEQ ID NO: 207 | SEQ ID NO: 67 | 47.1 | 4.2 |
| SEQ ID NO: 164 | SEQ ID NO: 4 | 46.8 | 2.9 |
| SEQ ID NO: 211 | SEQ ID NO: 51 | 46.3 | 1.4 |
| SEQ ID NO: 178 | SEQ ID NO: 18 | 45.6 | 0.6 |
| SEQ ID NO: 194 | SEQ ID NO: 34 | 45.0 | 6.5 |
| SEQ ID NO: 170 | SEQ ID NO: 10 | 44.2 | 0.9 |
| SEQ ID NO: 222 | SEQ ID NO: 62 | 44.0 | 2.4 |
| SEQ ID NO: 217 | SEQ ID NO: 57 | 43.9 | 2.9 |
| SEQ ID NO: 179 | SEQ ID NO: 19 | 43.3 | 0.9 |
| SEQ ID NO: 208 | SEQ ID NO: 48 | 42.9 | 1.3 |
| SEQ ID NO: 172 | SEQ ID NO: 12 | 42.3 | 1.8 |
| SEQ ID NO: 171 | SEQ ID NO: 11 | 41.7 | 0.2 |
| SEQ ID NO: 221 | SEQ ID NO: 61 | 41.3 | 2.2 |
| SEQ ID NO: 162 | SEQ ID NO: 2 | 40.6 | 2.4 |
| SEQ ID NO: 197 | SEQ ID NO: 37 | 40.5 | 9.4 |
| SEQ ID NO: 184 | SEQ ID NO: 24 | 40.4 | 4.1 |
| SEQ ID NO: 188 | SEQ ID NO: 28 | 40.4 | 3.6 |
| SEQ ID NO: 192 | SEQ ID NO: 32 | 40.1 | 3.4 |
| SEQ ID NO: 195 | SEQ ID NO: 35 | 40.1 | 4.6 |
| SEQ ID NO: 228 | SEQ ID NO: 68 | 39.9 | 1.3 |
| SEQ ID NO: 213 | SEQ ID NO: 53 | 39.5 | 1.2 |
| SEQ ID NO: 214 | SEQ ID NO: 54 | 39.4 | 2.3 |
| SEQ ID NO: 163 | SEQ ID NO: 3 | 39.2 | 2.9 |
| SEQ ID NO: 189 | SEQ ID NO: 29 | 38.9 | 2.4 |
| SEQ ID NO: 196 | SEQ ID NO: 36 | 38.3 | 0.6 |
| SEQ ID NO: 224 | SEQ ID NO: 64 | 37.7 | 1.4 |
| SEQ ID NO: 167 | SEQ ID NO: 7 | 37.5 | 2.8 |
| SEQ ID NO: 166 | SEQ ID NO: 6 | 37.1 | 5.3 |
| SEQ ID NO: 165 | SEQ ID NO: 5 | 36.8 | 3.4 |
| SEQ ID NO: 180 | SEQ ID NO: 20 | 36.0 | 1.6 |
| SEQ ID NO: 181 | SEQ ID NO: 21 | 35.9 | 2.9 |
| SEQ ID NO: 236 | SEQ ID NO: 76 | 35.6 | 0.5 |
| SEQ ID NO: 205 | SEQ ID NO: 45 | 35.2 | 1.4 |
| SEQ ID NO: 187 | SEQ ID NO: 27 | 33.0 | 6.2 |
| SEQ ID NO: 233 | SEQ ID NO: 73 | 32.7 | 0.6 |
| SEQ ID NO: 190 | SEQ ID NO: 30 | 31.9 | 0.6 |
| SEQ ID NO: 168 | SEQ ID NO: 8 | 31.3 | 3.5 |
| SEQ ID NO: 182 | SEQ ID NO: 22 | 29.2 | 0.5 |
| SEQ ID NO: 216 | SEQ ID NO: 56 | 26.5 | 4.2 |
| SEQ ID NO: 185 | SEQ ID NO: 25 | 22.9 | 5.3 |
| SEQ ID NO: 169 | SEQ ID NO: 9 | 21.6 | 0.9 |

^{a}Cleavage activity as measured by a T7E1 assay.

In particular, Cas9-NLS fusion proteins including NLS motifs having 10% or greater genome editing activity relative to a Cas9-NLS fusion protein having a SV40 NLS motif are preferred. Exemplary preferred NLS amino acid sequences include SEQ ID NO: 4, 11, 12, 13, 14, 15, 16, 17, 18, 19, 23, 26, 31, 33, 34, 38, 39, 40, 41, 42, 43, 44, 46, 47, 48, 49, 50, 51, 52, 55, 57, 58, 59, 60, 62, 63, 65, 66, 67, 69, 70, 71, 72, 74, 75, 77, 78, and 79. Even more preferred NLS amino acid sequences include SEQ ID NO: 15, 17, 33, 39, 40, 41, 42, 43, 44, 47, 49, 50, 52, 55, 58, 60, 63, 65, 71, 72, 74, 75, 77, 78, and 79. Highly preferred NLS amino acid sequences include SEQ ID NO: 41, 42, 43, 47, 63, 65, 71, 72, 74, and 75.

### Modularity and Portability of NLS Amino Acid Sequences to Other Proteins

Our previous work with CRISPR protein-NLS fusions demonstrated that NLS motifs have remarkable utility as modular cassettes for use in CRISPR proteins and other proteins generally. In this regard, a fusion protein having an NLS amino acid sequence can be configured in which the NLS amino acid sequence can be fused at the N-terminus or C-terminus of the target protein to effect nuclear localization. Additionally, a plurality of NLS amino acid sequences can be used in tandem or dispersed at the disparate locations within the target protein sequence to effect nuclear localization.

### Applicability of the Novel NLS Amino Acid Sequences to Other CRISPR Proteins.

Our previous work with CRISPR protein-NLS fusions strongly supports our view that the NLS amino acid sequences of the present invention should improve nuclear localization of CRISPR proteins bearing our new NLS motifs. We therefore predict that such CRISPR protein-NLS fusion proteins would exhibit robust genome editing following introduction into cells with a competent gRNA. An example of such CRISPR proteins serving as additional CRISPR protein-NLS fusion protein candidates include Acidaminococcus sp. CRISPR/Cas12a (Cpf1) protein.

### Applications

In a first aspect, an amino acid sequence including a nuclear localization signal (NLS) is provided. The amino acid sequence of the NLS includes a member selected from the group consisting of SEQ ID NO: 4, 11, 12, 13, 14, 15, 16, 17, 18, 19, 23, 26, 31, 33, 34, 38, 39, 40, 41, 42, 43, 44, 46, 47, 48, 49, 50, 51, 52, 55, 57, 58, 59, 60, 62, 63, 65, 66, 67, 69, 70, 71, 72, 74, 75, 77, 78, and 79.

In a first respect, the amino acid sequence of the NLS is selected from the group consisting of SEQ ID NO: 15, 17, 33, 39, 40, 41, 42, 43, 44, 47, 49, 50, 52, 55, 58, 60, 63, 65, 71, 72, 74, 75, 77, 78, and 79. In a second respect, the amino acid sequence of the NLS is selected from the group consisting of SEQ ID NO: 41, 42, 43, 47, 63, 65, 71, 72, 74, and 75.

In a second aspect, a fusion protein including a first amino acid sequence and a second amino acid sequence is provided. The second amino acid sequence includes a nuclear localization signal (NLS), wherein the NLS comprises a member selected from the group consisting of SEQ ID NO: 4, 11, 12, 13, 14, 15, 16, 17, 18, 19, 23, 26, 31, 33, 34, 38, 39, 40, 41, 42, 43, 44, 46, 47, 48, 49, 50, 51, 52, 55, 57, 58, 59, 60, 62, 63, 65, 66, 67, 69, 70, 71, 72, 74, 75, 77, 78, and 79.

In a first respect, the NLS includes a member selected from the group consisting of SEQ ID NO: 15, 17, 33, 39, 40, 41, 42, 43, 44, 47, 49, 50, 52, 55, 58, 60, 63, 65, 71, 72, 74, 75, 77, 78, and 79. In a second respect, the NLS includes a member selected from the group consisting of SEQ ID NO: 15, 17, 33, 39, 40, 41, 42, 43, 44, 47, 49, 50, 52, 55, 58, 60, 63, 65, 71, 72, 74, 75, 77, 78, and 79. In a third respect, the second amino acid sequence is positioned at the N-terminus of the first amino acid sequence or wherein the second amino acid sequence is positioned at the C-terminus of the first amino acid sequence. In a fourth respect, a plurality of the second amino acid sequence is present in the fusion protein. In a fifth respect, the first amino acid sequence comprises SEQ ID NO: 159. In a sixth respect, the first amino acid sequence comprises SEQ ID NO: 159 and wherein the second amino acid sequence is positioned at the N-terminus of the first amino acid sequence or wherein the second amino acid sequence is positioned at the C-terminus of the first amino acid sequence, and/or wherein a plurality of the second amino acid sequence is present in the fusion protein.

In a third aspect, an isolated Cas9-NLS fusion protein is provided. The Cas9-NLS fusion protein includes a Cas9 amino acid sequence and a nuclear localization signal sequence (NLS). The isolated Cas9-NLS fusion protein is active in a CRISPR/Cas endonuclease system, wherein the isolated Cas9-NLS fusion protein comprises a member selected from the group consisting of SEQ ID NO: 162-239.

In a fourth aspect, a nucleic acid sequence encoding a nuclear localization signal (NLS) is provided. The nucleic acid sequence encoding the NLS includes a member selected from the group consisting of SEQ ID NO: 83, 90, 91, 92, 93, 94, 95, 96, 97, 98, 102, 105, 110, 113, 117, 118, 119, 120, 121, 122, 123, 125, 126, 127, 128, 129, 130, 131, 134, 136, 137, 138, 139, 141, 142, 144, 145, 146, 148, 149, 150, 151, 153, 154, 156, 157, and 158.

In a first respect, the nucleic acid sequence encoding the NLS includes a member selected from the group consisting of SEQ ID NO: 94, 96, 112, 118, 119, 120, 121, 122, 123, 126, 128, 129, 131, 134, 137, 139, 142, 144, 150, 151, 153, 154, 156, 157, and 158. In a second respect, the nucleic acid sequence encoding the NLS includes a member selected from the group consisting of SEQ ID NO: 120, 121, 122, 126, 142, 144, 150, 151, 153, and 154.

In a fifth aspect, an isolated nucleic acid sequence encoding the isolated Cas9-NLS fusion protein is provided. In a first respect, the isolated nucleic acid sequence includes a member selected from the group consisting of SEQ ID NO: 241-318.

In a sixth aspect, an isolated ribonucleoprotein complex is provided. The isolated ribonucleoprotein complex includes a Cas9-NLS fusion protein and a gRNA.

In a first respect, the gRNA includes a crRNA and a tracrRNA in stoichiometric (1:1) ratio. In a second respect, the crRNA includes an Alt-R® crRNA (Integrated DNA Technologies, Inc. (Skokie, IL (US)) directed against a specific editing target site for a given locus and the tracrRNA includes Alt-R® tracrRNA (Integrated DNA Technologies, Inc. (Skokie, IL (US)). In a third respect, the gRNA includes a sgRNA.

In a seventh aspect, a CRISPR/Cas endonuclease system is provided. The CRISPR/Cas endonuclease system includes an isolated Cas9-NLS fusion protein. The isolated Cas9-NLS fusion protein includes a member selected from the group consisting of SEQ ID NO: 162-239.

In a first respect, the CRISPR/Cas endonuclease system is encoded by a DNA expression vector having a member selected from the group consisting of SEQ ID NO: 241-318. In a second respect, the DNA expression vector is a plasmid-borne vector. In a third respect, the DNA expression vector is selected from a bacterial expression vector and a eukaryotic expression vector.

In an eighth aspect, a method of performing gene editing in a eukaryotic cell is provided. The method includes a step of contacting a candidate editing target site locus with an active CRISPR/Cas endonuclease system having a Cas9-NLS fusion protein. The Cas9-NLS fusion protein includes a member selected from the group consisting of SEQ ID NO: 162-239.

In a ninth aspect, a kit for performing gene editing in a eukaryotic cell is provided. The kit includes a Cas9-NLS fusion protein, optionally, a nucleic acid encoding a Cas9-NLS fusion protein, and, optionally, a gRNA. The Cas9-NLS fusion protein includes a member selected from the group consisting of SEQ ID NO: 162-239. Where included in the kit, the nucleic acid encoding a Cas9-NLS fusion protein includes a member selected from the group consisting of SEQ ID NO: 241-318.

The applications of Cas9-based tools are many and varied. They include, but are not limited to: plant gene editing, yeast gene editing, mammalian gene editing, editing of cells in the organs of live animals, editing of embryos, rapid generation of knockout/knock-in animal lines, generating an animal model of disease state, correcting a disease state, inserting a reporter gene, and whole genome functional screening.

### Example 1

Cas9-NLS Fusions Delivered by Plasmid into HEK293 Cells Yields a Spectrum of Editing Efficiencies

The following example demonstrates that SpCas9-NLS fusion proteins with a wide variety of different NLS sequences demonstrates a wide spectrum of editing efficiencies in human cells relative to a SpCas9-SV40 NLS control (see FIG. 1). The results indicate that the ideal NLS sequence for SpCas9 is not obvious, and that a highly efficient SpCas9 genome editing solution must be empirically determined as was done in this study. SpCas9-NLS fusion proteins were tested using guides that target the HPRT-38087 locus in human cells.

### Example 2

Purified Cas9-NLS Fusions Delivered as RNP Complexes into HEK293 and K562 Cells Yields Improved Editing Efficiencies Over Prior Art Cas9-NLS Fusion Protein Commercial Options

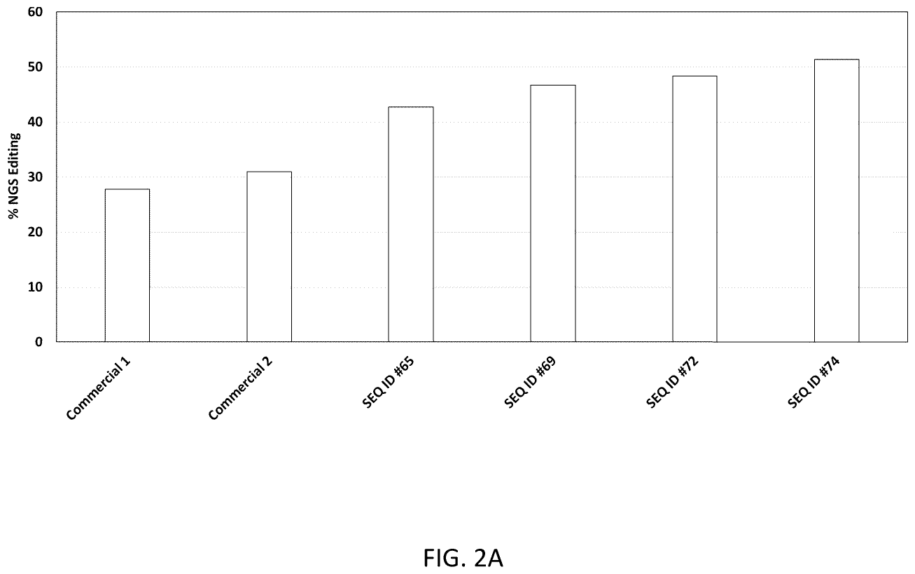

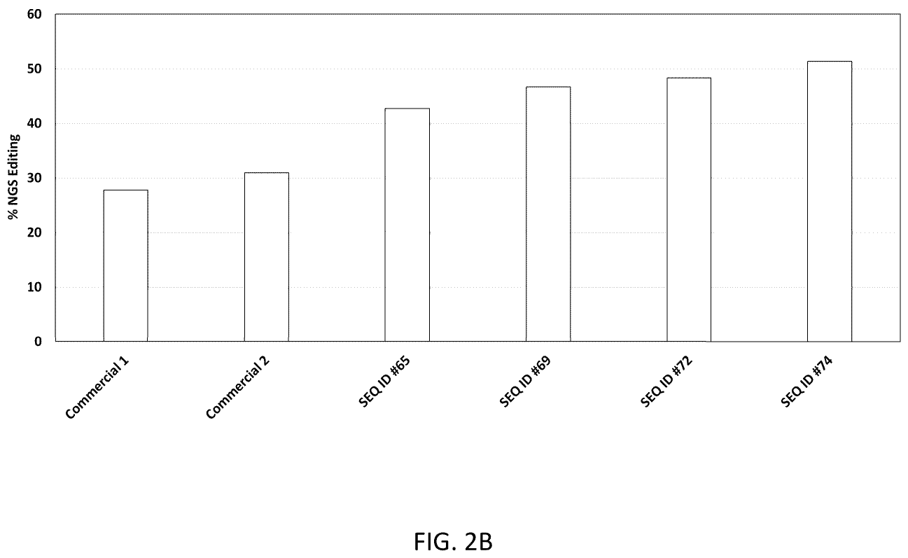

The following example demonstrates that purification and RNP delivery of the most active SpCas9-NLS fusion proteins (from Example 1) provides superior editing efficiencies over prior art Cas9-NLS fusion protein commercial options when delivered into either HEK293 or K562 cells (see FIG. 2A and FIG. 2B, respectively). The results indicate that the ideal NLS sequence for SpCas9 is not obvious, and that a highly efficient SpCas9 genome editing solution must be empirically determined as was done in this study. SpCas9-NLS fusion proteins were tested using guides that target the HPRT-38285 locus in human cells.

### Example 3

### Amino Acid and Nucleic Acid Sequences

The following amino acid and nucleic acid sequences support this disclosure.

Lengthy table referenced here
US20260103695A1-20260416-T00001
Please refer to the end of the specification for access instructions.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

LENGTHY TABLES
The patent application contains a lengthy table section. A copy of the table is available in electronic form from the USPTO web site (https://seqdata.uspto.gov/docdetail?docId=US20260103695A1). An electronic copy of the table will also be available from the USPTO upon request and payment of the fee set forth in 37 CFR 1.19(b)(3).

1. An amino acid sequence comprising a nuclear localization signal (NLS), wherein amino acid sequence of the NLS comprises a member selected from the group consisting of SEQ ID NO: 4, 11, 12, 13, 14, 15, 16, 17, 18, 19, 23, 26, 31, 33, 34, 38, 39, 40, 41, 42, 43, 44, 46, 47, 48, 49, 50, 51, 52, 55, 57, 58, 59, 60, 62, 63, 65, 66, 67, 69, 70, 71, 72, 74, 75, 77, 78, and 79.

2. The amino acid sequence of the NLS of claim 1, wherein amino acid sequence of the NLS is selected from the group consisting of SEQ ID NO: 15, 17, 33, 39, 40, 41, 42, 43, 44, 47, 49, 50, 52, 55, 58, 60, 63, 65, 71, 72, 74, 75, 77, 78, and 79.

3. The amino acid sequence of the NLS of claim 1, wherein amino acid sequence of the NLS is selected from the group consisting of SEQ ID NO: 41, 42, 43, 47, 63, 65, 71, 72, 74, and 75.

4. A fusion protein comprising a first amino acid sequence and a second amino acid sequence, wherein the second amino acid sequence comprises a nuclear localization signal (NLS), wherein the NLS comprises a member selected from the group consisting of SEQ ID NO: 4, 11, 12, 13, 14, 15, 16, 17, 18, 19, 23, 26, 31, 33, 34, 38, 39, 40, 41, 42, 43, 44, 46, 47, 48, 49, 50, 51, 52, 55, 57, 58, 59, 60, 62, 63, 65, 66, 67, 69, 70, 71, 72, 74, 75, 77, 78, and 79.

5. The fusion protein of claim 4, wherein the NLS comprises a member selected from the group consisting of SEQ ID NO: 15, 17, 33, 39, 40, 41, 42, 43, 44, 47, 49, 50, 52, 55, 58, 60, 63, 65, 71, 72, 74, 75, 77, 78, and 79.

6. The fusion protein of claim 4, wherein the NLS comprises a member selected from the group consisting of SEQ ID NO: 15, 17, 33, 39, 40, 41, 42, 43, 44, 47, 49, 50, 52, 55, 58, 60, 63, 65, 71, 72, 74, 75, 77, 78, and 79.

7. The fusion protein according to claim 4, wherein the second amino acid sequence is positioned at the N-terminus of the first amino acid sequence or wherein the second amino acid sequence is positioned at the C-terminus of the first amino acid sequence.

8. The fusion protein according to claim 4, wherein a plurality of the second amino acid sequence is present in the fusion protein.

9. The fusion protein according to claim 4, wherein the first amino acid sequence comprises SEQ ID NO: 159.

10. The fusion protein according to claim 4, wherein the first amino acid sequence comprises SEQ ID NO: 159 and wherein the second amino acid sequence is positioned at the N-terminus of the first amino acid sequence or wherein the second amino acid sequence is positioned at the C-terminus of the first amino acid sequence.

11. The fusion protein according to claim 10, wherein a plurality of the second amino acid sequence is present in the fusion protein.

12. An isolated Cas9-NLS fusion protein, wherein the Cas9-NLS fusion protein comprises a Cas9 amino acid sequence and a nuclear localization signal sequence (NLS), wherein the isolated Cas9-NLS fusion protein is active in a CRISPR/Cas endonuclease system, wherein the isolated Cas9-NLS fusion protein comprises a member selected from the group consisting of SEQ ID NO: 162-239.

13-15. (canceled)

16. An isolated nucleic acid sequence encoding the isolated Cas9-NLS fusion protein of claim 12.

17. The isolated nucleic acid sequence encoding the isolated Cas9-NLS fusion protein of claim 16, wherein the isolated nucleic acid sequence comprises a member selected from the group consisting of SEQ ID NO: 241-318.

18. An isolated ribonucleoprotein complex, wherein the isolated ribonucleoprotein complex comprises a Cas9-NLS fusion protein of claim 12 and a gRNA.

19. The isolated ribonucleoprotein complex of claim 18, wherein the gRNA comprises a crRNA and a tracrRNA in stoichiometric (1:1) ratio.

20. The isolated ribonucleoprotein complex of claim 19, wherein the crRNA comprises an Alt-R® crRNA directed against a specific editing target site for a given locus and the tracrRNA includes Alt-R® tracrRNA.

21. The isolated ribonucleoprotein complex of claim 19, wherein the gRNA comprises a sgRNA.

22-27. (canceled)
